(19) United States
(12) Patent Application Publication
(10) Pub. No.: US 2009/0118816 A1
(43) Pub. Date: May 7, 2009

(54) Implantable Device for Therapeutic Treatment Within a Body Lumen

(75) Inventors: Nicholas Kipshidze, New York, NY (US); Gary Lichttenegger, Woodinville, WA (US); Alexander J. Pallenberg, Duvall, WA (US); Nick Yeo, Great Bookham (GB); Thomas L. Wenger, Durham, NC (US)

Correspondence Address: SEED INTELLECTUAL PROPERTY LAW GROUP PLLC, 701 FIFTH AVE, SUITE 5400, SEATTLE, WA, 98104, US

(73) Assignee: Light Sciences Oncology, Inc.

(21) Appl. No.: 11/991,589

(22) PCT Filed: Sep. 5, 2006
(86) PCT No.: PCT/US2006/034607

§ 371 Date: Jan. 7, 2009

Related U.S. Application Data
(60) Provisional application No. 60/714,314, filed on Sep. 6, 2005.

(51) Int. Cl. A61F 2/06

(52) U.S. Cl. 623/1.34; 623/1.42

(57) ABSTRACT

A treatment system to provide energy therapy to a patient includes an implantable device and an activation system having a plurality of energy emitters that transmit energy towards the device. In one embodiment, the implantable device is a stent carrying a treatment agent. The treatment agent can be selectively activated by the activation system before, during, or after implantation in a subject's body. The activated treatment agent can react with adjacent tissues.

### BACKGROUND

1. Field

The present disclosure generally relates to treatment systems, devices, and methods usable for medical treatment and, more particularly, to systems, devices, and methods employing energy therapy for the treatment of a body lumen.

2. Description of the Related Art

Typical photodynamic therapy (“PDT”) employs light to treat or investigate photosensitized tissues. A photoreactive or photosensitizing agent having a characteristic light absorption waveband is typically administered to the patient, either orally or by injection or even by local delivery to the treatment site. The photoreactive or photosensitizing agent is subsequently selectively absorbed by abnormal tissue much more so than by normal tissue. Once the abnormal tissue has absorbed or linked with the photoreactive or photosensitizing agent, the abnormal tissue can then be destroyed by administering light of an appropriate wavelength or waveband corresponding to the absorption wavelength or waveband of the photoreactive agent. PDT has proven effective in destroying abnormal tissue, such as cancer cells. Traditional PDT is often unsuitable for performing highly localized treatments on, for example, vascular targets largely because typical methods of delivering the photoreactive agent do not permit accurate localization of the PDT.

Atherosclerotic cardiovascular diseases is typically treated by performing percutaneous transluminal coronary angioplasty (PTCA) to relieve narrowing or stenosis of an artery by atherosclerotic plaque. Drug eluting stents are often used to reduce the rate of restenosis following PTCA. Intravascular stent devices are often coated with or partially comprised of cytotoxic or other anti-proliferative compounds. These compounds are released into the vessel surface when exposed to aminopeptidases, carboxypeptidases, endopeptidases, other proteolytic enzymes, as well as other naturally occurring chemical constituents of the body. Unlike typical delivery of photosensitive agents, the stent releases the compounds locally. The rates of drug release and dose delivery are dependent upon the properties of the linking moiety used to attach the compound to the stent. Drug eluting stents while effectively overcoming the problem of restenosis caused by unwanted proliferation of vessel wall tissue in and around the deployed stent have a disadvantage in that continued elution of the cytostatic or cytotoxic drug over a long period of time suppresses the normal healing response leaving the stent exposed to the blood and creating a potential site for clot formation. Additionally, drug eluting stents once deployed continue to release drug until it is exhausted and are then unable to control any further episode of proliferation that could jeopardize patency of the lumen and may ultimately lead to a requirement for further vascular intervention to deal with the even more morbid problem of in-stent restenosis.

The present disclosure is directed to overcome one or more of the above-mention difficulties, and provide further added benefits.

### BRIEF SUMMARY

In one aspect, the present disclosure is directed to a treatment device. The treatment device may include an intraluminal stent configured for placement in a body lumen to preserve flow through that lumen. The intraluminal stent may include an energy-activated therapeutic agent that forms, at least in part, a surface of the intraluminal stent.

In another aspect, the present disclosure is directed to a stent device. The stent device includes an implantable elongate body and a chemically active material. In some embodiments, the stent is dimensioned for placement in a body lumen. The chemically active material may include a sufficient amount of energy-activatable therapeutic agent for effectively treating target tissue of a body lumen in which the elongate body is placed when the therapeutic agent is activated by applied energy. In some embodiments, the chemically active material is coupled to the elongate body.

In another aspect, the present disclosure is directed to a system for treating a subject. The system includes a stent device and an activation system. The stent device includes a selectively activatable treatment agent that, when activated, ablates target tissue proximate the stent device. The activation system may be configured to output a sufficient amount of energy to activate a therapeutically effective amount of the treatment agent when the stent device is positioned in situ.

In another aspect, the present disclosure is directed to a method for treating a body lumen in a subject. The method includes implanting a stent in the body lumen, applying energy to the subject, and activating a therapeutically effective amount of the treatment agent with the applied energy. In some embodiments, the stent may include a stent structure dimensioned to closely fit within the body lumen, and a treatment agent coupled to the stent structure.

In yet another aspect, the present disclosure is directed to a stent device that includes an implantable body and a treatment agent. The implantable body is dimensioned for placement in a body lumen. In some embodiments, the treatment agent is coupled to the elongate body and includes a sufficient amount of an energy-activatable agent for effectively treating target tissue of the body lumen without being released in appreciable amounts when the therapeutic agent is activated by applied energy.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles may not be drawn to scale, and some of these elements may be arbitrarily enlarged and positioned to improve drawing legibility.

FIG. 1A is a side elevational view of a treatment system positioned in a body lumen, according to one illustrated embodiment.

FIG. 1B is a side elevational view of the treatment system of FIG. 1A, where an activation system of the treatment system emits energy towards an implanted stent of the treatment system, according to another illustrated embodiment.

FIG. 1C is a side elevational view of the treatment system of FIG. 1A after tissue has been treated, according to another illustrated embodiment.

FIG. 2 is a pictorial view of a treatment system for use in a body lumen, according to another illustrated embodiment.

FIG. 3 is an isometric view of an implantable device of the treatment system of FIG. 2.

FIG. 4 is a front elevational view of the implantable device of FIG. 3.

FIG. 5 is a cross-sectional view of the implantable device of FIG. 4 taken along line 5-5.

FIG. 6 is a side elevational view of an implantable device with anchors, according to another illustrated embodiment.

FIG. 7 is a cross-sectional view of a covering and a strut of an implantable device, according to another illustrated embodiment.

FIG. 8 is a cross-sectional view of a covering and a strut of an implantable device, according to another illustrated embodiment.

FIG. 9 is a cross-sectional view of a covering and a strut of an implantable device, according to another illustrated embodiment.

FIG. 10 is a cross-sectional view of a covering and a strut of an implantable device, where the covering has a plurality of medicaments, according to another illustrated embodiment.

FIG. 11 is a side elevational view of an implantable device, according to another illustrated embodiment.

FIGS. 12 to 16 are axial cross-sectional views of struts of an implantable device, where the struts are coated with a treatment agent, according to another illustrated embodiment.

FIG. 17 is a side elevational view of a delivery system carrying a collapsed implantable device having a treatment agent, according to another illustrated embodiment.

FIG. 18 is a side elevational view of the delivery system of FIG. 17, where the implantable device extends out of the delivery system, according to another illustrated embodiment.

FIG. 19 is a side elevational view of the delivery system of FIG. 18, where the implanted device is in an expanded configuration, according to another illustrated embodiment.

FIG. 20 is a side elevational view an activation system spaced from the implanted device of FIG. 19, according to another illustrated embodiment.

FIG. 21 is a side elevational view of the activation system positioned within the implanted device, according to another illustrated embodiment.

FIG. 22 is a side elevational view of the implanted device and surrounding tissue treated by energy therapy, according to another illustrated embodiment.

FIG. 23 is a side elevational view of a treatment system, where an external activation system delivers energy to an implanted device, according to another illustrated embodiment.

FIG. 24 is a side elevational view of the external activation system of FIG. 23, according to another illustrated embodiment.

FIG. 25 is a bottom elevational view of the external activation system of FIG. 23, according to another illustrated embodiment.

FIG. 26 is a side elevational view of a treatment system, where an activation system is outside a body lumen in which a stent device is implanted, according to another illustrated embodiment.

### DETAILED DESCRIPTION

The embodiments described herein are generally related to treatment systems usable for therapeutically treating one or more internal target sites. The treatment systems can include one or more implantable devices and an activation system. The implantable devices can have a therapeutic agent that is selectively activated by the activation system before, during, or after implantation in a subject's body. The therapeutic agent can be a treatment agent suitable for intraluminal use.

As used herein, the term “treatment agent” is a broad term and includes, but is not limited to, one or more energy-activatable substances used in therapy. Light, acoustic waves (e.g., ultrasound waves), combinations thereof, or any other applied energy is capable of activating the treatment agents described herein. Exemplary non-limiting treatment agents include, without limitation, one or more pharmaceutically active substances, photoreactive agents, photosensitizing agents, photodynamic compounds, activatable therapeutic agents, or combinations thereof.

The treatment system, employing at least one treatment agent, can perform one or more types of energy therapies, including, for example, light therapy, ultrasound therapy, acoustic therapy, and the like. The various energy therapies can be used to treat many types of medical conditions, such as for example, proliferative diseases including cancer, vascular diseases or conditions, abnormal tissues, and the like. In some embodiments, the various energy therapies can be performed without physically contacting the targeted tissues, thereby reducing or limiting trauma to the subject and recovery periods. To increase the efficiency of the treatment system, the treatment agent is directly contacted to the target tissue.

The implantable device of the treatment system can take the form of a stent provided with one or more non-eluting treatment agents, eluting treatment agents, energy activated treatment agents, combinations thereof, or other treatment agents. In non-eluting embodiments, the treatment agent can be administered when activated at a selected period (or periods) of time determined to be beneficial to the patient. Any number of discrete treatment cycles can be performed. In some embodiments, the non-eluting drug is not released in appreciable amounts. In some other embodiments, the non-eluting drug does not erode or degrade when in the body. In some other embodiments, the agent may remain coupled to the stent and activatable for a limited period of time or indefinitely.

In eluting embodiments, the treatment agent can be administered over a predetermined period (or periods) of time. In some embodiments, the agent is releasable somewhat continuously into the patient's blood stream. The release rate is selectable based on the patient's condition and desired drug dispersion. In some embodiments, the released drug and/or the drug still bound to the implanted device can be selectively activated. The released treatment agents dispersed in the subject can treat remote tissue, whereas the treatment agents attached to the implanted device can treat tissue proximate the implanted device.

The stents can take the form of intraluminal stents, intravascular stents, non-vascular stents, or other type of stents. A stent can maintain the desired shape of a body lumen. For example, a stent can open or maintain a flow passageway of a body lumen after performing a procedure, such as a debulking procedure, that may cause narrowing of the lumen. In some embodiments, the stent can restore a body lumen obstructed by, for example, plaque (e.g., atherosclerotic plaque), diseased tissue, a tumor, proliferated tissue, and other vessel narrowing conditions. For example, tissue can grow inwardly through the frame of the stent due to cell proliferation triggered, at least in part, by the presence of the stent. In some embodiments, energy therapy can either reduce the amount or cause regression of the proliferative tissue, thereby restoring proper fluid flow through the stent.

There are many types of obstructive conditions that can cause undesirable closing or narrowing of body lumens. For example, obstructions may form in the gastrointestinal tract (e.g., esophageal obstructions, gastric or duodenal obstructions, pancreatico-biliary obstructions, colorectal obstructions, and the like), genitourinary tract, broncho pulmonary passageways, and the like. The cardiovascular system may often have atherosclerotic obstructions, thrombotic occlusions, clots, or restenosis following traditional endovascular interventions. To treat these conditions, stents can be implanted to restore proper functioning of affected body lumens.

The stents can be coated with or contain as part of their chemically accessible surface structure, one or more therapeutic treatment agents (e.g., compounds such as a photodynamic agents, mono-L-aspartyl chlorine e-6, and the like) that, when activated by energy, produces a reaction that inhibits, reduces, delays, or substantially prevents flow-limiting or constrictive intraluminal diseases (such as restenosis), cancerous cell proliferation, arterial plaque buildup, and the like. In some embodiments, the treatment agent is overlying, coating, embedded in, absorbed in, or combinations thereof at least a portion of the stent and arranged for provision to a subject. The stent's outer surface, in whole or in part, can be formed of, the treatment agent, for example. This accessible outer surface can biologically interact with tissue of a body.

The activation system can be a light generating catheter for insertion into and delivery through a body lumen in which the device carrying the treatment agent is implanted. The light generating catheter can include a light source array containing light emitting diodes (“LEDs”) disposed on conductive traces electrically connected to leads extending proximally through a lumen of the light generating catheter to an external power supply and control device. In addition to LEDs, other sources of light may be used, such as, organic LEDs, super luminescent diodes, laser diodes, or light emitting polymers. For example, applicant's co-pending patent application U.S. Publication No. 2005/0228260 (U.S. patent application Ser. No. 10/799,357); U.S. Pat. No. 5,800,478; and U.S. Pat. No. 7,018,395 disclose various types of light emitting devices and elements that can be utilized in the activation system. Each of these references is incorporated by reference in its entirety. For example, an endovascular catheter capable of outputting light of the appropriate absorptive wavelength can activate the treatment agent.

The treatment agent can include a chemically active surface that retains the photodynamic agent for an extended period after implantation during which PDT may be performed. In some embodiments, the treatment agent has a plurality of states, such as an inert state, an activated state, a highly active state, and/or reactive state. The agent may remain inert before the activation process. Such inert agents may not significantly affect the subject's body. For example, inert treatment agents may be benign agents that do not cause any appreciable physiological changes due to any biological interactions with, for example, tissue in close proximity to the inert agent. An activation process can transform the inert agent into a highly active material that causes biological interactions with the subject. The activation period can correspond to a beneficial treatment period.

The treatment system can treat various tissues in a subject's body, including, without limitation, diseased or abnormal tissues (e.g., cancerous cells), interstitial tissues, epithelial tissues, connective tissues (e.g., cartilage, bone, and the like), nerve tissues, blood, or other regions of interest. The number, sizes, and configurations of the implantable devices of the treatment system can be selected based on the tissue to be treated. Treatment agents may be selected based on their sensitivity to stimuli (e.g., sensitivity to applied energy). Treatment agents may also be selected based in part on the location and depth of the implantation site. In some other embodiments, the treatment agent's may be imparted with a particular sensitivity to stimuli (e.g., sensitivity to applied energy) based in part on the location and depth of the implantation site. To facilitate thorough activation of a desired amount of the treatment agent, for example, the sensitivity of the treatment agent can be increased the deeper the implantation site.

To activate the treatment agent, energy can be applied from within and/or external to the subject's body. To internally apply energy, minimally invasive delivery techniques can be used to reduce the risk of infection, internal bleeding, and other complications often associated with surgical procedures. Unlike surgical tissue removal producers (e.g., surgical procedures for removing cancerous tissue), tissue destroyed by energy therapy can be naturally absorbed by the body. Thus, energy therapy can be performed relatively quickly without using complicated tissue removal instruments.

External activation systems can direct and deliver energy to internally implanted devices. Recovery times can be significantly reduced because energy is applied without gaining internal access to the implanted device. Energy transmitted from the external activation system to the implanted device may pass harmlessly through non-targeted tissue.

In some diagnostic applications, the activation system of the treatment system can emit light with a wavelength selected to cause photo-reactive treatment agent to fluoresce as a means to acquire information about the targeted cells without damaging the targeted cells. For example, an implantable device carrying the photoreactive agent can be positioned adjacent cells to be diagnosed. The photoreactive agent can be activated any number of times to monitor the patient's condition.

In some therapeutic applications, the wavelength of the light delivered causes the treatment agent to undergo a photochemical reaction with oxygen in the targeted cells proximate the agent, to yield free radical species (such as singlet oxygen), which causes localized cell destruction (e.g., cell lysis), through apoptosis, or necrosis, for example. This can be performed with or without physically contacting the target tissue with the agent. To treat an artery, for example, PDT can inhibit arterial restenosis by causing a depletion of vascular smooth muscle cells, which can be a source of neointima cell proliferation (see, Nagae et al., Lasers in Surgery and Medicine 28:381-388, 2001). One of the advantages of PDT is that it is a targeting therapy. Preferential localization of a photoreactive treatment agent in areas of an arterial injury compared to non-injured arterial wall can allow highly specific PDT ablation.

In other embodiments, the activation system outputs ultrasound waves. An ultrasound activation system for internal use can be an intraluminal catheter with one or more transducers that selectively output ultrasound waves. Such activations systems can be navigated through body lumens of the patient.

An external activation system can be held against or proximate the subject while it delivers ultrasound waves into the subject. The ultrasound waves propagate through the patient to the implanted stent and activate the treatment agent. The external ultrasound activation system, in some embodiments, can be a handheld device for placement against the subject's skin.

Various access techniques can be used to deliver the implantable devices. Open procedures, semi-open procedures, laparoscopic procedures, and minimally invasive procedures (e.g., percutaneous techniques) can provide suitable access to the target delivery site. Known conventional surgical instruments (e.g., sizing rings, balloons, calipers, gauges, delivery sheaths, catheters, tubes, cannulas, and the like) can be used to access the deployment sites. Many times, the access techniques and procedures can be performed by the surgeon and/or a robotic device, such as robotic systems used for performing minimally invasive surgery. Those skilled in the art recognize that there are many different ways to access internal deployment sites.

The treatment systems disclosed herein can be used to treat different subjects. As used herein, the term “subject” generally refers to any host, animal, and particularly humans. The terms “patient” and “subject” are used interchangeably herein.

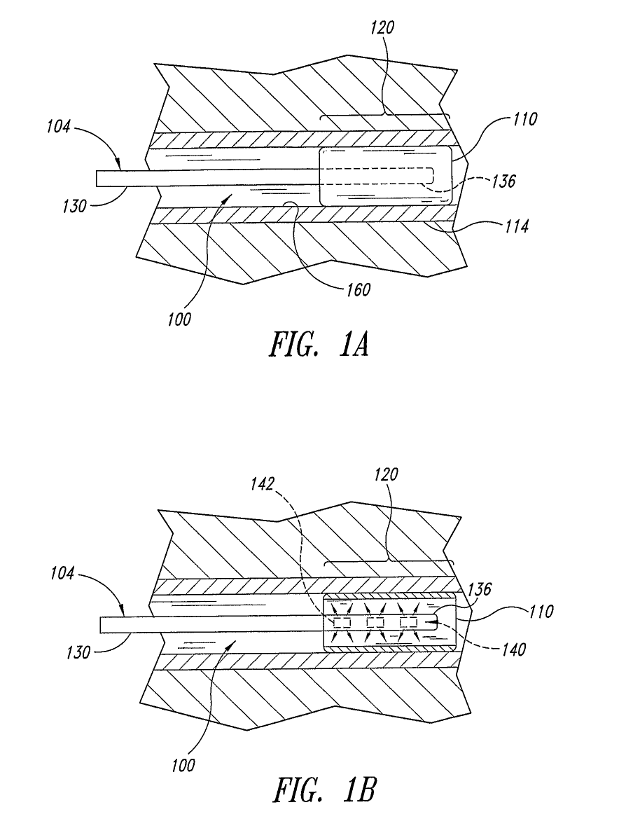

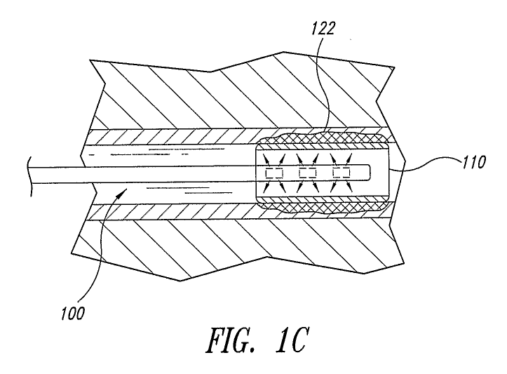

As shown in FIGS. 1A through 1C, the illustrated treatment system 100 includes an activation system 104 and an intraluminal device 110 in a body lumen 114. The intraluminal device 110 carries one or more treatment agents that can be controllably activated by energy outputted from the activation system 104. To treat a target site 120, the device 110 can be positioned in the lumen 114 in proximity to the target site 120. The treatment agent, when activated, provides localized therapy on the cells of the target site 120 without appreciably affecting remote tissues. Different types of energy can activate the treatment agent.

With continued reference to FIG. 1B, energy from the activation system 104 activates the treatment agent of the device 110 (shown in cross-section). The agent then biologically interacts with at least a portion of the cells of the adjacent target site 120. The biological reaction, in some embodiments, causes localized cell destruction, cell lysis, cell size reduction, necrosis, or combinations thereof. The target site 120 may have cancerous cells, abnormal tissues, proliferation diseased cells, and the like. In some embodiments, the activated treatment agent reacts with and destroys cancerous cells. Accordingly, unwanted cells, in proximity to the device 110, can be accurately destroyed in a controlled manner. Energy can be applied to the treatment agent until the desired amount of tissue 122 (FIG. 1C) has been treated.

FIG. 1C shows the treated tissue 122 surrounding the implanted device 110. If the treated tissue 122 is destroyed cells, the dead tissue 122 can be absorbed by the surrounding tissue, or discharged from the subject's body. Additional treatments can be performed at a later time to treat additional tissue at the target site 120. The illustrated implanted device 110 can be used numerous times to repeatedly treat the target site 120.

FIG. 2 shows the treatment system 100 including the activation system 104 and implantable device 110. The activation system 104 is in the form of a catheter having an elongate catheter body 130 coupled to a controller 132. A distal tip 136 of the elongate catheter body 130 carries an energy emission system 140 (shown in phantom). A power source 133 (shown in phantom) can be electrically coupled to the energy emission system 140.

The illustrated energy emission system 140 is a plurality of energy emitters 142 embedded within the distal tip 136. Each of the emitters 142 can output outwardly directed energy that activates at least a portion of the surrounding treatment agent carried by the device 110. As used herein, the term “energy emitter” is a broad term and includes, but is not limited to, an energy output device capable of selectively activating a treatment agent. The illustrated energy emitters 142 can remotely activate treatment agent for improved flexibility when selecting an appropriate treatment procedure. Once the emitters 142 are within range of the device 110, the energized emitters 142 can output a sufficient amount of energy to activate the agent, even though the emitters 142 are spaced from the device 110.

The energy generated by the emitters 142 can be radiant energy (e.g., electromagnetic energy), mechanical energy (e.g., acoustic energy such as ultrasound waves), combinations thereof, or other types of energy that can be controllably delivered through a subject. To minimize or limit damage to non-targeted tissue, the applied energy can pass freely through the non-targeted tissue, whereas targeted tissue interacts with the treatment agent. Non-limiting exemplary energy emitters can be light sources, transducers, wave generators, sound generators, or combinations thereof.

The illustrated treatment system 100 can be configured to perform light therapy. The term “light therapy” as used herein is broadly construed to include, without limitation, photo-activating or photo-exciting one or more target cells by subjecting a target area or site (e.g., one or more target cells) to one or more wavelengths of light. The wavelengths of light can be approximately close to, if not equivalent to, at least one excitation wavelength of the target cells, for example. This photo-excitation process can be used during an oncology treatment program, for example, to treat diseased cells, cancerous target cells, or otherwise undesirable cells. For cardiovascular treatment programs, the photo-excitation process can treat diseased vascular tissue, plaque (e.g., vulnerable plaque), thrombosis (e.g., coronary thrombosis), atherosclerosis, restenosis, hyperplasia, or other target tissues to improve the health of the cardiovascular system. Hyperplasia can be due to a chronic inflammatory response, hormonal dysfunctions, and medical treatments (e.g., iatrogenic injury caused by procedures for treating coronary artery disease, procedures for treating peripheral artery disease of the carotids or the lower extremities, and the like) or following the placement of a vascular graft (e.g., to create vascular access in patients undergoing hemodialysis).

It is understood that even if one cell type is “targeted,” it is possible that other cell types in a vicinity of the targeted cell may also be subjected to therapy. The therapy, however, may be focused on the target site to minimize collateral damage. During light therapy, for example, cells positioned away from the implanted device 110 may be destroyed; however, the treatment effect can be concentrated at the target site 120 (FIGS. 1A and 1B). If therapy is used to destroy built up plaque along artery walls, some of the cells of the artery wall may likewise be destroyed.

The emitters 142 in the form of light sources can be capable of outputting visible light waves, non-visible light waves, and combinations thereof. The energy sources can be LEDs (such as edge emitting LEDs, surface emitting LEDs, super luminescent LEDs), laser diodes, lasers, or other light sources capable of outputting light suitable for performing light therapy on the subject. The illustrated energy emitters 142 of FIG. 1B can emit radiation wavelength(s) or waveband(s) that corresponds with, or at least overlap with, the wavelength(s) or waveband(s) that excite or otherwise activate the agent. Photosensitive treatment agents can often have one or more absorption wavelengths or wavebands that excite them to produce substances which damage, destroy, or otherwise treat target tissues of the patient. For example, the energy emitters 142 can be configured to emit light having a wavelength or waveband in the range from about 400 nanometers to 1,000 nanometers. In some embodiments, the emitters 142 output a wavelength or waveband in the range from about 600 nanometers to about 800 nanometers. In some embodiments, the emitters 142 output a wavelength or waveband in the range from about 600 nanometers to about 700 nanometers. In one embodiment, for example, the emitters 142 output radiation with a peak wavelength of 664 nanometers plus or minus 5 nanometers. Other wavelengths are also possible. The treatment agent's wavelength of activation can be selected based on the optical properties of any intermediate substances (such as tissues, fluids, and the like) between the activation system 104 and stent 110. The proximity requirement to activate the treatment agent can be determined, at least in part, by the treatment agent's wavelength of activation and these intermediate substances.

With continued reference to FIG. 2, the controller 132 is coupled to a proximal end 146 of the elongate catheter body 130 and has a user input device 150 for adjusting the output of the emission system 140. The illustrated input device 150 is a rotatable dial that can be operated to control the level of emitted energy. Other types of analog or digital input devices can also be used. A switch 152 is used to turn the emission system 1400N and OFF.

The device 110 of FIG. 2 is in the form of an intraluminal stent. As used herein, the term “stent” is a broad term and includes, but is not limited to, a tube or tube-like structure that can be implanted in a patient. Intraluminal stents are one type of stent suitable for insertion into a lumen of an anatomical vessel. The anatomical vessel can be part of the vascular system, respiratory system, or other system, organ, or tissue suitable for undergoing therapy treatment. For example, stents can treat constrictive conditions that cause undesirable narrowing or blocking of the arteries. In some vasculature procedures, the stent is placed in a coronary artery after performing balloon angioplasty or another typical vasculature treatment. In this manner, conditions (e.g., restenosis) associated with vasculature treatments can be minimized, limited, or substantially prevented. In some biliary system procedures, a stent is placed across a stricture in a bile duct in order to maintain the proper flow of bile through the bile duct and may also be placed to ameliorate the occlusion of the bile duct by cancer (cholangiocarcinoma).

The device 110 can advantageously perform multiple functions for a more efficacious treatment. For example, the device 110 can hold open a body lumen, control fluid flow, or perform other functions traditionally performed by stents, as well as performing light therapy. An endovascular stent, sometimes referred to as a “stent graft,” can be used to reinforce weak or damaged lumens. These types of stents can include fabric supported by a metal mesh. These stents can, for example, reinforce a weak spot (e.g., an aneurysm) in an artery.

The body lumen in which the device 110 is placed can be, without limitation, a vessel, duct, canal, or other anatomical vessel or structure having a passageway for transporting fluids. Stents may be useful in maintaining proper fluid flow through the trachea, bronchial passageways, vasculature passageways, for example. Stents in some embodiments can open an air passageway that is narrowed or blocked by a tumor. In other embodiments, stents are used to treat ruptured vessels, aneurysms, or other conditions often associated with anatomical vessels.

The device 110 can also comprise one or more medicaments that may or may not be related to the energy therapy to be performed. The medicaments (including eluting and/or non-eluting medicaments) may not be “activatable” treatment agents and, consequently, can function independently of the activatable treatment agents. To reduce or limit tissue growth, for example, the device 110 may include a passive growth inhibitor (e.g., a non-photosensitive growth inhibitor, proliferation inhibitors, vascular cell growth inhibitors, such as PDGF inhibitors, Trapidil, cytotoxin, and the like) to limit, minimize, or substantially prevent cell proliferation. The energy activated treatment agent can destroy tissue and the growth inhibitor can limit new tissue growth. In this manner, the treatment agent and growth inhibitor work in combination to effectively eliminate or limited unwanted tissue growth.

In other embodiments, the device 110 may comprise one or more growth promoters that facilitate cell proliferation. The energy activated treatment agent can destroy unwanted abnormal tissue leaving substantially health, normal tissue. The growth promoter can then stimulate growth of this healthy tissue. A treatment program can include repeatedly destroying undesirable tissues and promoting growth of healthy tissue resulting in the rapid elimination of wanted tissue and reendothelialization of healthy, normal tissue.

The activation of the treatment agent can facilitate in the release of a secondary medicament (e.g., one or more non-photosensitive agents). When the treatment agent is activated, a ligand binding the secondary medicament to the device 110 is cleaved. The secondary medicament is then suitable for local action and/or regional action. Other types of release mechanisms can also be used to release the secondary medicament at a desired rate.

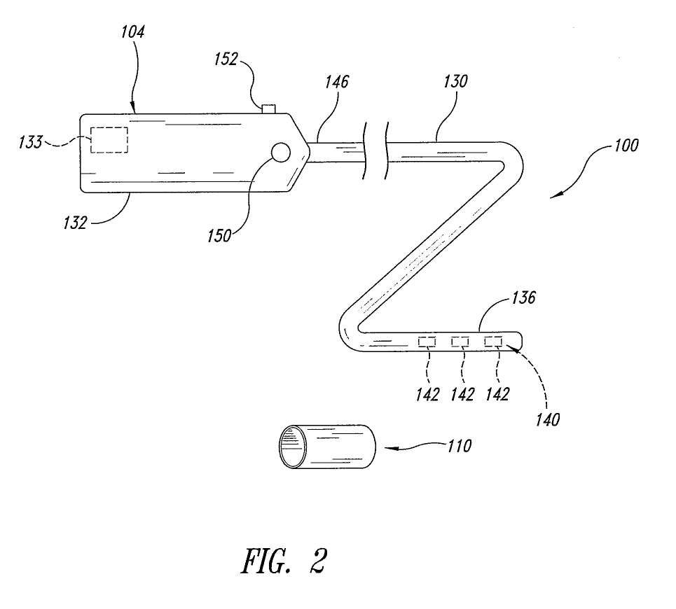

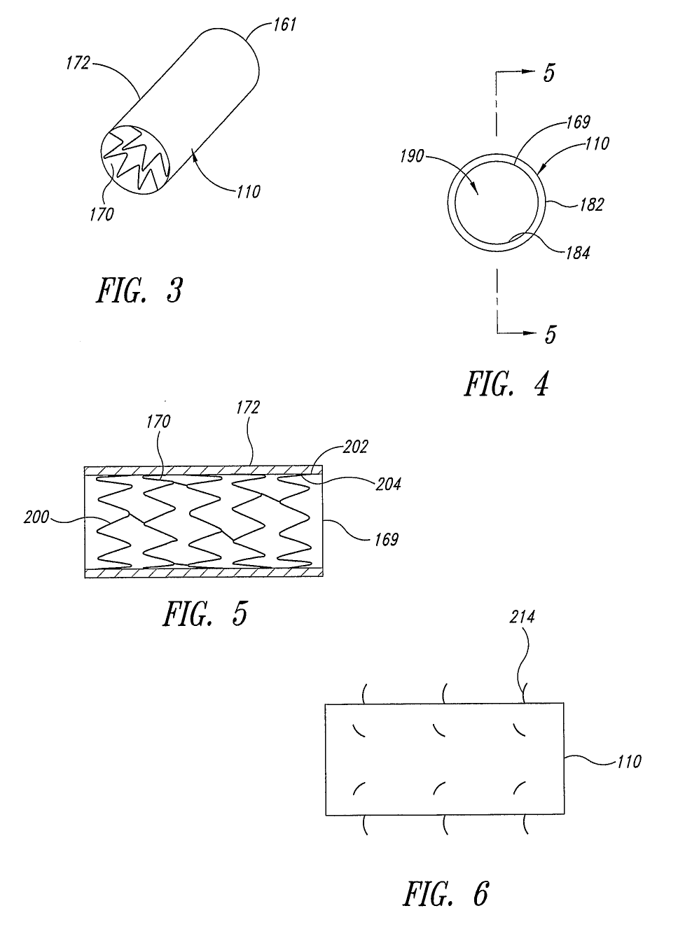

With reference to FIGS. 3 to 5, the implantable device 110 has an elongate body 169 including a frame 170 and a covering 172 coupled to the frame 170. The device 110 can have a collapsed state for delivery and an expanded state for anchoring to the lumen 114, as shown in FIGS. 1A to 1C. The device 110 can be either self-expanding or non-self-expanding. In some self-expanding embodiments, the frame 170 is made from a shape memory material, which can move the device 110 between a collapsed configuration and an expanded configuration when activated. The shape memory material may include, for example, a shape memory alloy (e.g., NiTi), a shape memory polymer, ferromagnetic material, or other material. These materials can be transformed from a first preset configuration to a second preset configuration when activated (e.g., thermally activated). In some embodiments, the device 110 self-expands when it is delivered out of a delivery system, such as a delivery catheter or restraining sheath. The frame 170 expands due to spring force following the device 110 being positioning within the lumen, after a restraining sheath is retracted from the compressed device 110.

In non-self-expanding embodiments, the device 110 can be expanded using an expansion balloon, inflatable angioplasty balloon, or other expansion device. Additionally or alternatively, the frame 170 can be made of metal, steel (e.g., stainless steel), plastic, combinations thereof, or other biocompatible materials. As discussed below, the frame 170 may comprise transparent materials to permit the delivery of light therethrough.

With reference to FIG. 4, the device 110 defines an outer surface 182 and an inner surface 184 opposing the outer surface 182. The outer surface 182 can be formed, in whole or in part, of a treatment agent. Direct contact between the agent and tissue ensures rapid and effective treatment without the inefficiencies associated with transmitting energy through an intermediate structure or across a gap.

The inner surface 184 of the device 110 defines a passageway 190 for fluid flow therethrough. The passageway 190 is dimensioned to receive the distal tip 136 of the activation system 104 and permit delivery of energy along the length of the device 110, as shown in FIG. 1B. The length of the portion of the distal tip 136 extending through the device 110 can correspond to the length of the treatment site 120. Accordingly, the activation system 104 can aim energy at the portion of the device 110 contacting the targeted tissue. In this manner, the activation system 104 accurately treats specifically targeted cells thereby limiting collateral damage of healthy non-targeted tissue.

The frame 170 of FIGS. 3 and 5 is a flexible mesh formed by a plurality of struts 200, each having a somewhat zig-zag configuration. The covering 172 can be attached to the outer surfaces 202 or inner surfaces 204 of the frame 170. Alternatively, the frame 170 can be embedded in the covering 172.

The illustrated covering 172 is coupled to the outer surfaces 202 of the frame 170. The covering 172 can be fabric, a membrane, or other material that can be coupled to the frame 170. A therapeutically effective amount of treatment agent can be disposed on or in the covering 172. The amount of emitted energy can be selected to activate a desired amount of the therapeutic agent, thereby treating a desired amount of targeted tissue.

As noted above, the activation system 104 may be capable of generating light. In such embodiments, the device 110 can be optically transparent to facilitate illumination of the treatment agent. Light can pass through the device 110 to the treatment agent, which in turn treats the target site. As shown in FIG. 1B, outwardly directed light is transmitted through the covering 172 until it reaches the treatment agent. The light then activates the treatment agent disposed along the outer surface 182. The transparent device 110 permits activation of the treatment agent, which can remain in generally continuous contact with the target tissue 120.

Suitable transmissive materials include, but are not limited to, polymers such as polyester, PET, polypropylene, combinations thereof and the like. These materials can form, in whole or in part, the frame 170 and/or covering 172. A substantial amount of the light directed from the activation system 104 towards the device 110 can be transmitted through the device 110. In some embodiments, at least 40% of the light emitted towards the covering 172 is transmitted therethrough. In some embodiments, at least 50% of the light directed towards the covering 172 is transmitted therethrough. In some embodiments, at least 60% of the light directed towards the covering 172 is transmitted therethrough. In some embodiments, at least 70% of the light directed towards the covering 172 is transmitted therethrough. In some embodiments, at least 80% of the light directed towards the covering 172 is transmitted therethrough. In some embodiments, at least 90% of the light directed towards the covering 172 is transmitted therethrough. In some embodiments, at least 95% of the light directed towards the covering 172 is transmitted therethrough.

The covering 172 optionally includes one or more opaque materials that can inhibit or prevent one or more wavelengths or wavebands from passing therethrough. Opacification agents, additives, coatings, or combinations thereof can be utilized to render the covering (or portion thereof somewhat opaque. In some embodiments, the opacification agents include, but are not limited to, dyes, pigments, metal particulates or powder, or other materials that can be coated onto, disbursed throughout, or otherwise disposed in the covering 172. If desired, the covering 172 can function as a filter so as to inhibit or prevent one or more wavelengths or wavebands from reaching the patient's tissue.

The illustrated device 110 has a generally circular axial profile that can generally match the axial profile of the body lumen into which it is to be placed. For example, the device 110 for use in the coronary can have a diameter less than about 3 mm, 4 mm, 5 mm, or 6 mm. The device 110 for use in the biliary system can have a slightly larger diameter. For example, a biliary device 110 can have a diameter less than about 5 mm, 7 mm, 9 mm, or 13 mm. In other embodiments, the device 110 can have a non-circular profile, especially if the device 110 is to be implanted in non-circular passageways (e.g., elliptical passageways).

The treatment agent, in some embodiments, can be attached to the device 110 and not released in appreciable amounts. In such embodiments, the treatment agent generally does not erode, degrade, or elute when implanted within the body. The treatment agent can be activated via application of energy from within, or external to the body, and produce the desired reaction while remaining attached to the device 110. The phrase “not released in appreciable amounts” as used herein generally means, without limitation, that a sufficient amount of the therapeutic agent of the device remains in place and accessible to the surrounding medium to allow activation with energy that produces a reaction that inhibits, reduces, delays, or prevents flow-limiting intraluminal or constrictive diseases. The treatment agent, in some embodiments, forms a chemically accessible surface and is not released in appreciable amounts.

The treatment agent can remain bound to the stent 110 for an extended period of time or indefinitely and can remain pharmologically inert before, during, and/or after absorption of applied energy. Advantageously, because the treatment agent is not destroyed by the activation energy, one or more additional treatments can be performed without administering additional treatment agents. Thus, at least some the problems associated with drug delivery (e.g., via intravenous injection) are reduced. Systemic drug delivery, for example, can cause photosensitizing of non-targeted tissue, which then may then be destroyed during subsequent PDT. The stent 100, in contrast, provides localized drug administration for reducing the amount of photosensitized non-targeted tissues.

In some embodiments, in accordance with the teachings described below, at least 50% by weight of the treatment agent remains bound to the device 110 and activatable 12 months after implantation in a subject. The treatment agent can be used to perform effective therapies even after long term implantation. In some embodiments, at least 90% by weight of the treatment agent remains bound and activatable 6 months after implantation the subject. After implantation, additional therapy treatments may need to be performed within 6 months. Because the treatment agent is effectively bound to the device 110, the target site can be treated when needed. In some embodiments, at least 30% by weight of the treatment agent remains bound and activatable 12 months after implantation. In yet other embodiments, at least 50% by weight of the treatment agent remains bound and activatable 24 months after implantation. A subject's condition may require additional follow up procedures 24 months after implantation. For example, cancer may recur years after an initial treatment or progress gradually, but in either case there is potential to cause lumen obstruction as a source of morbidity or mortality. Advantageously, the device 110 can remain at a target site for years to enable convenient follow up procedures.

To limit or substantially prevent migration, the device 110 can have one or more anchors, rougheners, or other anchoring means that interacts with the wall 160 of the lumen 114 of FIG. 1A. Exemplary anchors include, without limitation, barbs, spikes, protrusions, hooks, or other structures for piercing or otherwise interacting with the lumen wall 160. FIG. 6 shows the device 110 with a plurality of anchors 214. Other types of fixation features can also be used to implant the device 110.

The term “implant” as used herein is a broad term and includes, without limitation, to insert and leave in living tissue for a threshold amount of time. The implantable device 110 of FIGS. 1A to 1C can be inserted into and left in living tissue for an extended period of time. In some embodiments, the device 110 is fixedly implanted by using surgical techniques. The term “fixedly implant” as used herein includes, but is not limited to, inserting and anchoring a device, including stents or stent-like devices, to tissue. Fixedly implanted stents can remain coupled to tissue over a long period of time. Frictional interaction between the stent and tissue may be sufficient to fixedly implant the stent. Of course, anchors 214 can fixedly implant the device 110.

Additionally or alternatively, the device 110 can have one or more valves (e.g., one-way valves, two-way valves, duck bill valves, and the like), flow restrictors or regulators, or other flow regulating means to control the flow of fluid (e.g., air, blood, bile, urine, and the like) through the device 110. These components can be releasably or permanently coupled to the frame 170 of the device 110.

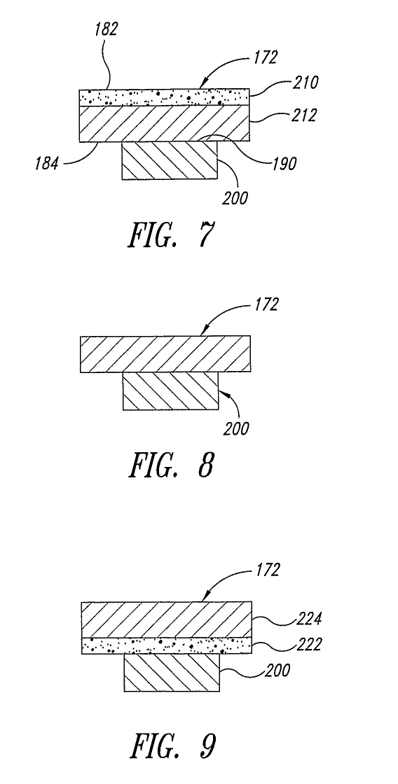

FIGS. 7 to 10 show various embodiments of coverings that can be incorporated into the stent device 110. FIG. 7 shows the cover 170 coupled to the strut 200. The illustrated inner surface 184 is coupled (e.g., adhered, bonded, or otherwise affixed) to the outer surface 190 of the strut 200.

The illustrated cover 170 has an outer layer 210 defining the outer surface 182 and an inner layer 212 defining the inner surface 184. The outer layer 210 can comprise a therapeutically effective amount of treatment agent. The outer layer 210 can be formed by a coating/deposition process. For example, treatment agent can be sprayed onto the inner layer 212 so as to build up and form the outer layer 210. In other embodiments, a preformed sheet is cut and then coupled to the inner layer 212 to form the outer layer 210. In yet other embodiments, a dipping process can be used to form the outer layer 210. Other deposition/coating processes (e.g., chemical vapor deposition) can form the outer layer 210 having a desired thickness.

In some embodiments, one or more linking moieties can be employed to link the outer layer 210 to the inner layer 212 (e.g., a polymer layer). In some embodiments, the linking moieties can be selected to achieve a desired rate of release of the treatment agent. In some embodiments, the linking moieties are selected such that the treatment agent is not released in appreciable amounts. The linking moieties may include one or more functional groups selected from amino acids, including, without limitation, enantiomers of naturally occurring L-amino acids such as D-amino acids. The amino acids may include naturally occurring and non-naturally occurring amino acids, and derivatives, isomers or combinations thereof. In some embodiments, the amino acids include functional groups or other structures not found in natural amino acids.

The therapeutic agent can be joined to the device, for example, via an amide-containing linking moiety consisting of species not readily acted upon by naturally-occurring enzyme systems that would otherwise be able to cleave the therapeutic agent from the device 110. The linking moiety can be comprised of appropriate combinations of D-amino acids, synthetic amino acids, sterically hindered structures, and other molecular components or functional groups designed to be somewhat resistant to the action of species occurring in living systems that could cause the separation of the therapeutic agent from the device. These species include, without limitation, proteolytic enzymes (e.g., carboxypeptidases, aminopeptidases, endopeptidases, and the like), and other naturally-occurring agents that can give rise to the undesired cleavage or hydrolysis reactions.

One chemical structure of a treatment agent, including a linking moiety, of the device 110 is represented by the following Formula 1:

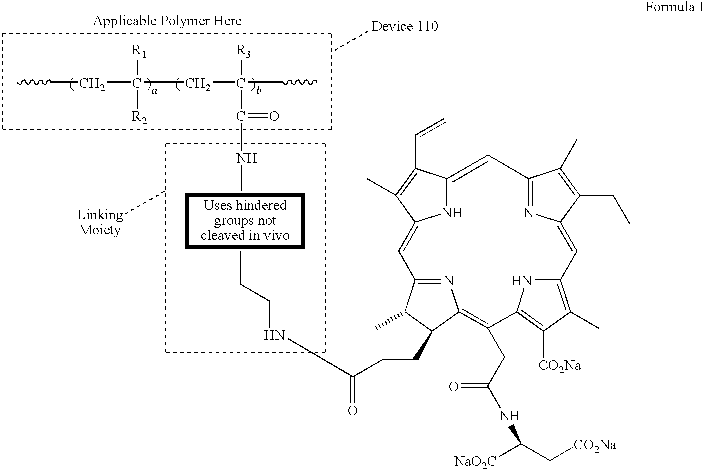

This treatment agent can remain on the device 110 for an extended period of time. The rate of release can be controlled by selecting a linking moiety that keeps the rate of cleaving at or below a desired rate. The linking moiety can contain one or more amide functional groups, and can include, without limitation, one or more molecules or substituent not readily acted upon by naturally-occurring enzyme systems that would otherwise be able to cleave the therapeutic agent from the device. Other types of moieties or linking compounds can be selected to achieve the desired linking to the device 110.

The linking moiety can also include compounds having a shape or steric bulk that hinders, limits or substantially prevents them from being acted upon by enzymes, including, without limitation, naturally occurring enzymes which include, but are not limited to, aminopeptidases, carboxypeptidases, endopeptidases, and other proteolytic enzymes. In some embodiments, the linking moiety may include straight or branched, saturated or unsaturated, chain alkyl groups having 1 to 25 carbon atoms. The linking moiety alky groups may optionally comprise at least one double bond and/or at least one triple bond. The linking moiety alkyl groups may further be unsubstituted or substituted in one or more positions with various groups. For example, such linking moiety alkyl groups may be optionally substituted with at least one group independently selected from alkyl, alkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, aralkoxycarbonylamino, halogen, alkyl thio, alkylsulfinyl, alkylsulfonyl, hydroxy, cyano, nitro, amino, monoalkylamino, dialkylamino, haloalkyl, haloalkoxy, aminoalkyl, monoalkylaminoalkyl, dialkylaminoalkyl, and the like. Additionally, at least one carbon within any such alkyl may be optionally replaced with —C(O)—, Sulfur, or Nitrogen. The linking moiety alky groups may be use to couple a treatment agent to the inner layer 212.

The treatment agent can be selected on its physicochemical characteristics. The treatment agent in some embodiments can comprise talaporfin sodium ((+)-tetrasodium (2S,3S)-18-carboxylato-20-[N—(S)-1,2-dicarboxylatoethyl]-carbamoylmethyl-13-ethyl-3,7,12,17-tetramethyl-8-vinylchlorin-2-propanoate). To apply the talaporfin treatment agent to the stent 110, an amide can be linked to the polymer cover 170. For example, an amide of talaporfin is formed using 1,3-diaminopropane in a regio-chemically defined process that occurs exclusively at the propionic acid side chain (i.e., the side chain at C-2, which is on the D-ring). This creates a mono-amino species linkable to a biocompatible polymer of the device 110 via diimide coupling using, for example, water-soluble EDAC reagents. If the stent 110 has hydroxyl groups or amino groups, then the talaporfin can be coupled directly under suitable pH conditions. The amino groups can provide enhanced stability because its amide groups can be more stable than, for example, esters. The polymer material forming, in whole or in part, the covering 172 or frame 170 can be selected based in part on the treatment agent and linking moieties.

FIGS. 8 to 10 show coverings that are similar to the covering 172 of FIG. 7, except as detailed below. FIG. 8 shows a covering 172 with an embedded treatment agent. The treatment agent can be evenly or unevenly disbursed throughout the covering 172. Various types of binders or fillers can provide the desired physical properties (e.g., elasticity, strength, barrier characteristics, and the like). Because the entire covering 172 comprises treatment agent, the device 110 can carry a relatively large amount of treatment agent.

FIG. 9 illustrates the covering 172 with an inner layer 222 comprising a treatment agent and an outer layer 224. The outer layer 224 can be formed of a biocompatible material for contacting tissue, thus forming a barrier between the inner layer 222 and tissue. That is, the outer layer 224 can limit or substantially prevent interaction (e.g., biological interaction) between the treatment agent and tissue. Upon activation, however, the treatment agent in the inner layer 222 can treat the tissue contacting, or near, the covering 172.

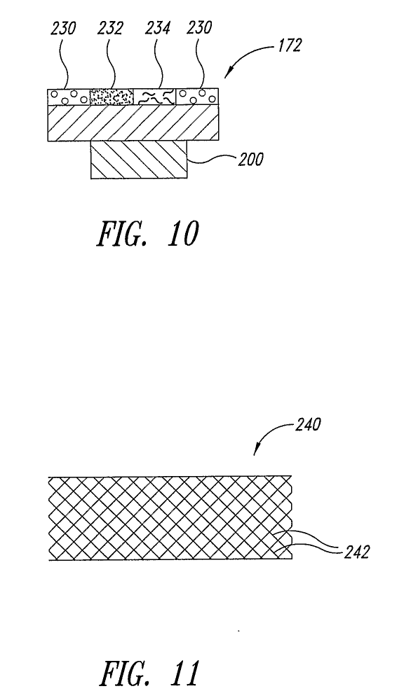

FIG. 10 illustrates the covering 172 including a plurality of medicaments 230, 232, 234. The medicaments 230, 232, 234 can each comprise a treatment agent, each automatable with the same or different energy. Thus, the medicaments 230, 232, 234 can be activated simultaneously or sequentially. In other embodiments, at least some of the medicaments 230, 232, 234 can comprise non-activatable medicaments, such as growth factors, growth inhibitors, radioactive materials, or combinations thereof. The medicaments 230, 232, 240 can form annular bands, longitudinal strips extending along the length of the device 110, or other patterns.

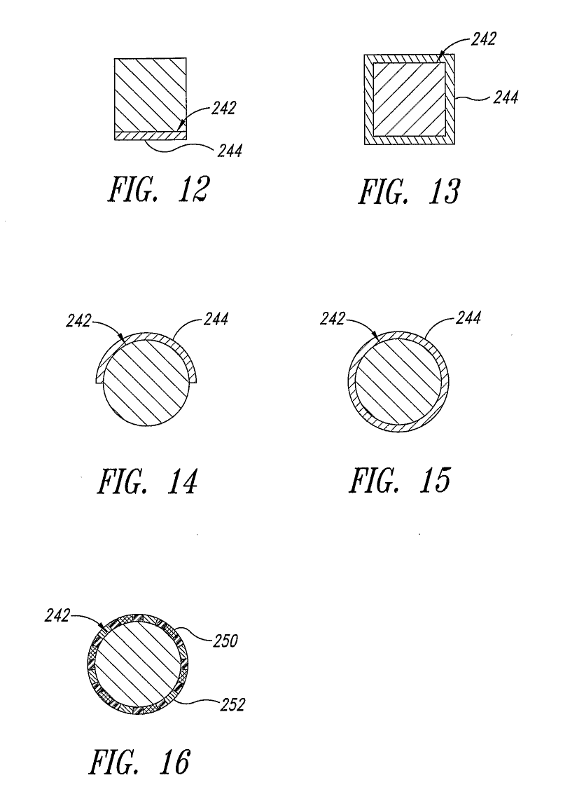

The frame of the implantable device can also carry the treatment agent. FIG. 11 shows a self-expanding frame 240 coated with a treatment agent. FIGS. 12 to 16 are cross-sectional views of one of the struts 242 of the frame 240. FIG. 12 illustrates the strut 242 with a generally polygonal axial cross-section, illustrated as a generally square cross-section, with at least one side coated with a treatment agent 244. FIG. 13 shows the treatment agent 244 encasing the strut 242. FIGS. 14 to 16 illustrate the strut 242 with a generally circular axial cross-section. FIG. 14 shows a partially coated strut 242. The entire strut 242 of FIG. 15 is coated with the treatment agent 244.

The struts 242 of the frame 240 can be coated with more than one type of treatment agent. The illustrated of strut 242 of FIG. 16 has a first treatment agent 250 and a second treatment agent 252 different than the first treatment agent 250. These treatment agents 250, 252 can be activated at the same time or different times. The agents 250, 252 can treat discrete regions of target tissue. The type, position, and amount of treatment agent can be selected based on the treatment to be performed.

In the embodiments illustrated in FIGS. 11 to 16 the frame 240 can comprise a transparent or semi-transparent material. As such, light transmitted through the frame 240 can activate the treatment agent. Additionally or alternatively, energy (e.g., light) can be conveniently transmitted through the gaps in the frame 240 for rapid and through activation of the treatment agent that may have been released from the frame 240.

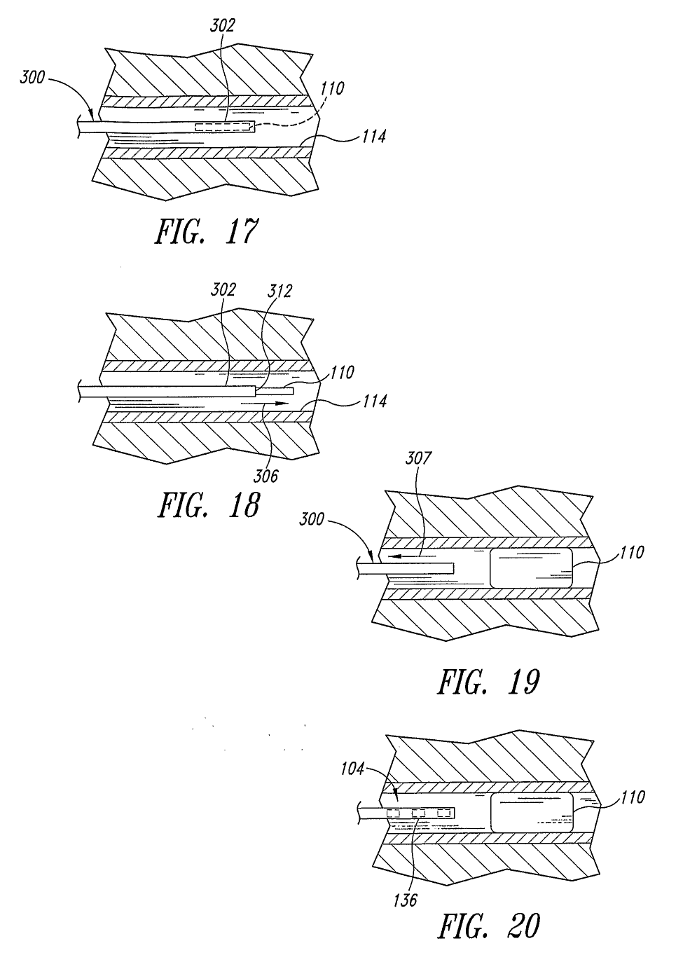

FIGS. 17 to 22 show one exemplary method using the treatment system 100 of FIG. 2. FIG. 17 shows a delivery system 300 positioned within the body lumen 114. The illustrated delivery system 300 is a delivery catheter having a distal tip 302 carrying the device 110 (shown in phantom in FIG. 17). The device 110 can be in a collapsed configuration to reduce the profile of the delivery system 300. The illustrated device 110 is in a compressed, collapsed state.

Various types of delivery systems can be used to deliver and deploy the device 110. For example, delivery sheaths, endoscopic instruments, trocars, guidewires, combinations thereof, or other delivery tools can be used.

With reference now to FIG. 18, once the distal tip 302 is in the desired position, the device 110 is pushed in the distal direction, as indicated by the arrow 306. As the device 110 moves out of the opening 312 of the distal tip 302, the device 110 expands against the body lumen 114 to anchor it in place. As noted above, the device 110 can optionally include one or more anchors for limiting, inhibiting, or substantially preventing migration in situ.

As shown in FIG. 19, once the device 110 is implanted, the delivery system 300 can be pulled proximally away from the implanted device 110 (indicated by the arrow 307) and subsequently removed from the patient. Alternatively, the delivery system 300 can be left in the patient and used to delivery the activation system 104. For example, the activation system 104 can be passed through a working lumen in the delivery system 300. Thus, a single system 300 can be used throughout the entire procedure.

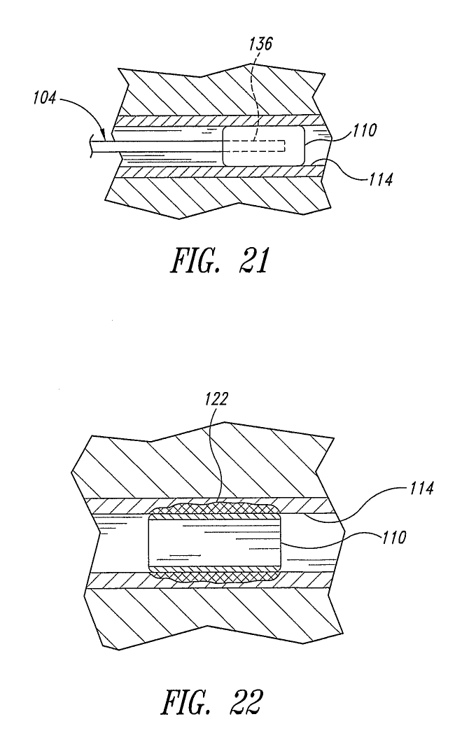

FIG. 20 shows the activation system 104 disposed within the body lumen 114 and spaced from the implanted device 110. The activation system 104 can be advanced distally until its distal tip 136 is positioned within the passageway 190 of the device 110, as shown in FIG. 21.

The treatment agent may be configured to biologically interact with the tissue or fluid that it is in contact with only upon being exposed to predefined amounts of energy. The agent can be either completely activated at a prescribed time period (such as when initially implanted within the body), or, because of the non-degrading nature of the agent, it can be activated after being implanted for extended time periods. The agent can also be configured in such a way that it could be activated repeatedly during a given time period.

Once the distal tip 136 is close enough to activate the treatment agent, the activation system 104 can emit energy for a desired treatment period. Because the activation system 104 can be spaced from the device 110, the likelihood of dislodging or moving the device 110 is reduced. Because the system 104 is close to the device 110, the treatment agent can absorb light with a wavelength less than about 800 nm. In some embodiments, the system 104 can activate the treatment agent so long as the system 104 is within several centimeters of the device 110. In some embodiments, the treatment agent absorbs wavelengths of light of in the range of about 630-670 nm for activation by the system 104 within at least 1 cm of the device 110. In some embodiments, the distal tip 136 may contact the implanted device 110 during the procedure. This may help maintain the proper position of the distal tip 136 relative to the device 110. After the target tissue has been treated, the delivery of energy can be stopped, and the activation system 104 can be retracted and removed from the subject.

FIG. 22 shows the treated tissue 122 surrounding the device 110. A layer of tissue 122 contacting or near the device 110 is substantially destroyed. The device 110 can be moved to another treatment site to perform additional procedures. Retractor instruments, repositioning instruments, or other types of intraluminal tools can be used to remove and/or reposition the device 110 for subsequent procedures. In diagnostic procedures, the tissue 122 of FIG. 22 can be tissues that fluoresce or otherwise become readily identifiable when subjected to applied energy.

The treatment agent can be configured with two layers of different compounds, each compound being molecularly distinct as to allow each layer to be activated at a different wavelength. For example, the device 110 can have the layers 210, 212 (see FIG. 7), wherein each of the layers 210, 212 is activated at a different wavelength or waveband. Thus, multiple layers can be designed to perform a series of treatments without removing and/or repositioning the device 110. This ensures that the device 110 remains at the treatment site for a desired length of time.

Activation of any treatment agent can take place any period of time during and/or after implantation. In some embodiments, the treatment agent is activated at least one day after implantation. The position of the device 110 can be monitored to ensure proper fixation within the body lumen 114. Then the treatment agent is activated. In some embodiments, the treatment agent is activated at least 1 month after implantation. Tissue may regrow leading to restenosis or re-narrowing of the lumen after the initial treatment. A subsequent procedure can be performed to treat (e.g., ablate) the new tissue. In some embodiments, the treatment agent is activated after at least 6 months, 12 months, and/or 24 months. Whether activated or not, the treatment agent can remain an inactive or passive constituent of the device 110 indefinitely.

If tissues grow through the frame 170, energy therapy can be performed to cause regression or destruction of the tissue that has grown between the frame and into the lumen. The treated tissue is resorbed into the surrounding tissue. In this manner, tissues extending inwardly into the lumen 190 can be conveniently removed.

Visualization techniques can be used to view the position of the device 110. Fluoroscopy (e.g., x-ray fluoroscopy), direct viewing, (e.g., laparoscopes, endoscopes, and the like), CT machines, angiography, or other suitable visualization systems or techniques can be used to view and determine the position of the device 110 in the patient in real time. The device 110 may include one or more markers (e.g., radio-opaque marks) viewable using the visualization techniques noted above. The number and positions of the markers may be selected based on the treatment to be performed. The markers can be made from a material readily identifiable after insertion into the patient's body. If x-ray fluoroscopy is employed, the markers can be made from gold or tungsten, for example. In some embodiments, the frame 170 is at least partially coated with gold or tungsten. Other types of markers can also be used.

The markers can also provide other information, such as the treatment agent carried by the device 110. Markers carrying different treatment agents can have different configurations. Thus, a physician can view markers to determine an appropriate means for activating the treatment agent, the precise positioning of the activation system prior to application of energy.

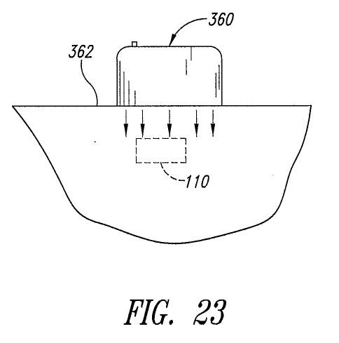

Externally applied energy can also be used to activate the treatment agents in a similar manner as the internally applied energy described above. FIG. 23 shows the implanted device 110 (shown in phantom) and an external activation system 360 that emits energy through the tissue 362 to the implanted device 110. As such, treatment agent on the device 110 can be activated without performing any surgical procedures, thus reducing risk of infection, tissue damage attributable to gaining physical access to the device 110, and overall recovery time.

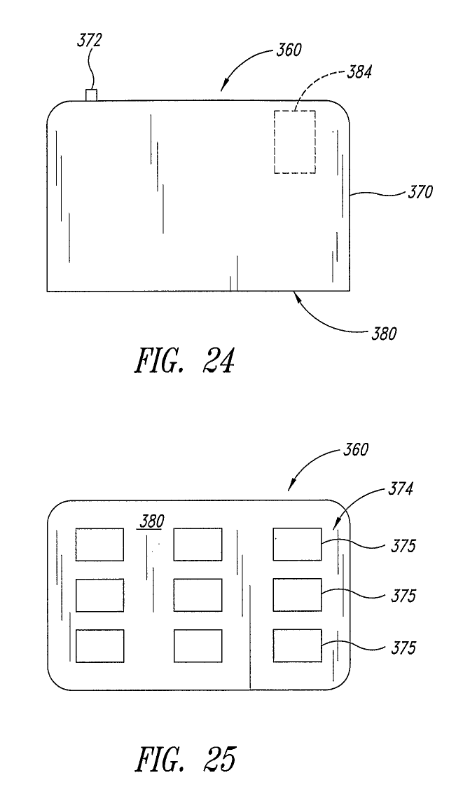

With reference to FIGS. 24 and 25, the external activation system 360 includes a main body or housing 370, a controller 372, and an energy emission system 374. The illustrated emission system 374 of FIG. 25 includes a plurality of energy emitters 375. When a bottom surface 380 of the main body 370 is placed against the tissue 362 (as shown in FIG. 23), the controller 372 can be used to operate the emission system 374, which emits energy that causes activation of the treatment agent of the device 110. The amount of energy can be increased or decreased based on the impedance of the tissue 362, distance to the device 110, and threshold energy level required to activate the treatment agent.

The number and position of the energy emitters 375 of the emission system 374 can be selected to generate the desired intensity and distribution of energy. If the energy emitters 375 are light sources, for example, they can be configured to generate a field of light that illuminates the device 110.

With reference to FIG. 24, the main body 370 includes a power supply 384 (shown in phantom) that powers the emission system 374. The controller 372 can be used to adjust the output from the emission system 374 and can be, for example, a rotatable dial. A user can conveniently and quickly set the energy output level.

The power supply 384 in the housing 370 can comprise one or more batteries. Alternatively, the system 360 can be powered by an AC power source, such as a typical AC electrical power outlet. Thus, various types of power sources can drive the components of the system 360.

In operation, the external delivery system 360 can be placed near or against the tissue 362. The system can be aimed towards the device such that energy travels through the tissue 362 to the implanted device 110. For maximized efficiency, the external delivery system 360 can be pressed against the tissue 362. After the desired amount of tissue is treated, the external system 360 can be turned OFF and separated from the subject. The system 360 can then be reused any number of times as desired.

In some embodiments, the plurality of energy emitters 375 of the system 374 can be transducers that emit mechanical energy, such as ultrasound waves. This mechanical energy can cause activation of the treatment agent with minimal adverse effects. Coupling media (e.g., gels, water, oil, creams, and the like) can facilitate transmission of the ultrasound waves to the tissue 362, which in turn transmits the ultrasound waves to the device 110. Similar to the light emitting systems described herein, the system 360 can emit ultrasound wave having one or more wavelengths at one or more frequencies.

The emitted waves may be, e.g., sinusoidal waves of defined amplitude, frequency, phase or waveshape, or may comprise pulses of defined amplitude and duration. The emission system 374 may comprise signal generators in which the control variables (amplitude, frequency, phase, pulse height, pulse duration, waveshape, etc.) are set by the user by manipulating the controller 372. Alternatively, emission system 374 may be responsive to a control program, stored either internally within the system 360 or externally to it, which defines and controls the desired parameters.

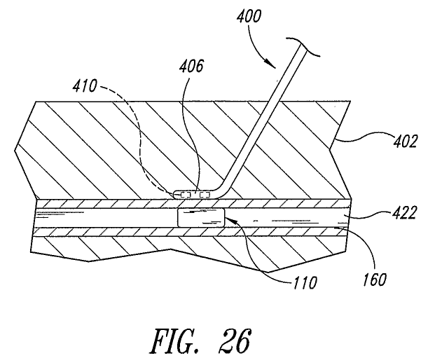

FIG. 26 illustrates an activation system 400 positioned near the implanted device 110. The delivery system 400 extends through the skin 402 and is at least proximate the implanted device 110. The activation system 400 has a distal tip 406 with an energy emission system 410 that delivers energy through the lumen wall 160 to the implanted device 110. Accordingly, the treatment agent can be conveniently activated without having to gain access to the passageway 422 of the lumen 114.

To insert the system 400 through the tissue 402, a delivery tool (e.g., a trocar, insertion needle, and the like) can form a delivery path suitable for placement of the distal tip 406. Alternatively, access can be provided by an open surgical procedure or through an incision (e.g. laparotomy) or the like. Activation of the treatment agent can be an adjunct procedure performed during the open surgical procedure.

The treatment agent of the stent 110 can have a longer wavelength of activation than the treatment agents of the stents activated with an intraluminal system, such as one of the intraluminal systems described above. The system 400 can be at a various locations outside of the body lumen 114. For example, if the device 110 is positioned in the coronary blood vessel, the system 400 can be located in another artery (e.g., the coronary sinus), adjacent vein, or other tissue or organ within range of the device 110. In some cardiovascular applications, the system 400 located outside of the heart and surrounding arteries can activate the device 110 in the coronary artery, thus avoiding any cardiovascular intervention.

Although implantable devices have been primarily described with light delivery systems utilizing transducers and LEDs, delivery systems having one or more lasers, fiber optic delivery systems, and/or optical elements can be used to accurately delivery light to a target region. Delivery of light from a source, such as a laser, to the treatment site can be accomplished through the use of a single, fiber optic delivery system with special light-diffusing tips affixed thereto. Some examples include a single fiber cylindrical diffuser, a spherical diffuser, a microlensing system, an over-the-wire cylindrical diffusing multi-fiber optic catheter, a light-diffusing fiber optic guidewire, etc.

All of the above U.S. patents, U.S. patent application publications, U.S. patent applications, foreign patents, foreign patent applications and non-patent publications referred to in this specification and/or listed in the Application Data Sheet, to include U.S. Pat. Nos. 6,958,498; 6,784,460; 6,661,167; and 6,445,011; U.S. Publication Nos. 2005/0228260 and 2005/0085455; International Patent Application Nos. PCT/US2005/032851 and PCT/US01/44046, are incorporated herein by reference, in their entireties. For example, the catheters and light systems disclosed in U.S. Pat. Nos. 5,800,478 and 5,766,234 can be used to activate the treatment agent. Except as described herein, the embodiments, features, systems, devices, materials, methods and techniques described herein may, in some embodiments, be similar to any one or more of the embodiments, features, systems, devices, materials, methods and techniques described in the incorporated references. In addition, the embodiments, features, systems, devices, materials, methods and techniques described herein may, in certain embodiments, be applied to or used in connection with any one or more of the embodiments, features, systems, devices, materials, methods and techniques disclosed in the above-mentioned incorporated references.

The various methods and techniques described above provide a number of ways to carryout the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments disclosed herein. Similarly, the various features and acts discussed above, as well as other known equivalents for each such feature or act, can be mixed and matched by one of ordinary skill in this art to perform methods in accordance with principles described herein. Additionally, the methods which are described and illustrated herein are not limited to the exact sequence of acts described, nor are they necessarily limited to the practice of all of the acts set forth. Other sequences of events or acts, or less than all of the events, or simultaneous occurrence of the events, may be utilized in practicing the embodiments of the invention.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. The materials, methods, ranges, and embodiments disclosed herein are given by way of example only and are not intended to limit the scope of the disclosure in any way. Accordingly, the invention is not intended to be limited by the specific disclosures of preferred embodiments disclosed herein.

What is claimed is:

1. A treatment device comprising:
an intraluminal stent configured for placement in a body lumen, the stent having a surface comprising one or more energy-activated therapeutic agents.

2. The device of claim 1 wherein the one or more energy-activated therapeutic agents are selected from photodynamic agents.

3. The device of claim 2, further comprising a tubular covering, the one or more energy-activated therapeutic agents coupled to the tubular covering of the device.

4. The device of claim 1 wherein the one or more energy-activated therapeutic agents are coupled by a linking moiety to the intraluminal stent, the linking moiety selected to substantially prevent enzymatic cleavage of the one or more energy-activated therapeutic agents from the device.

5. The device of claim 4 wherein the linking moiety couples the one or more energy-activated therapeutic agents to the intraluminal stent, wherein the linking moiety takes the form of a peptide linkage comprising one or more amino acids, wherein the one or more amino acids are selected from enantiomers of naturally occurring L-amino acids and D-amino acids.

6. The device of claim 4 wherein the linking moiety comprises one or more derivatives of naturally occurring amino acids, wherein the one or more derivates of naturally occurring amino acids are selected from isomers of naturally occurring amino acids, and naturally occurring amino acids substituted with one or more functional groups.

7. The device of claim 4 wherein the linking moiety comprises a secondary structure or steric conformation that substantially prevents the linking moiety from enzymatic cleavage by one or more enzymes selected from proteolytic enzymes, carboxypeptidases, endopeptidases, and aminopeptidases.

8. The device of claim 4 wherein the linking moiety is selected from one or more C₁-C₂₅ straight or branched, saturated or unsaturated, substituted or unsubstituted alkyl groups.

9. The device of claim 4 wherein the linking moiety is selected such that at least 50% by weight of the one or more energy-activated therapeutic agents remains coupled to the intraluminal stent after about 12 months implantation in a subject.

10. The device of claim 1 wherein at least 90% by weight of the one or more energy-activated therapeutic agents remains bound and activatable 6 months after implantation in a subject.

11. The device of claim 1 wherein at least 90% by weight of the one or more energy-activated therapeutic agents remains bound and activatable 12 months after implantation in a subject.

12. The device of claim 1 wherein at least 90% by weight of the one or more energy-activated therapeutic agents remains bound and activatable 24 months after implantation in a subject.

13. The device of claim 1 wherein at least 50% by weight of the one or more energy-activated therapeutic agents remains bound and activatable 6 months after implantation in a subject.

14. The device of claim 1 wherein at least 50% of the one or more energy-activated therapeutic agents remains bound and activatable 12 months after implantation in a subject.

15. The device of claim 1 wherein at least 50% of the photodynamic agent remains bound and activatable 24 months after implantation in a subject.

16. A stent device comprising:
an implantable elongate body dimensioned for placement in a body lumen; and
a chemically active material coupled to the elongate body, the chemically active material comprising a sufficient amount of energy-activatable therapeutic agent for effectively treating target tissue of a body lumen in which the elongate body is placed when the therapeutic agent is activated by applied energy.

17. The stent of claim 16 wherein the energy-activatable therapeutic agent is chemically linked to the elongate body.

18. The stent of claim 16 wherein the elongate body comprises a covering coupled to a generally tubular expandable frame, and the chemically active material forms a coating on the covering.

19. The stent of claim 16 wherein the energy-activatable therapeutic agent is a photodynamic agent capable of destroying tissue of the body lumen.

20. The stent of claim 16 wherein the energy-activatable therapeutic agent becomes reactive with the target tissue after activation.

21. The stent of claim 16 wherein the energy-activatable therapeutic agent has an unactivated state and an activated state for destroying tissue of the body lumen.

22. The stent of claim 21 wherein at least a portion of the energy-activatable therapeutic agent is adapted to remain in the unactivated state in situ until applied energy causes a therapeutically effective amount of the energy-activatable therapeutic agent to change from the unactivated state to the activated state.

23. The stent of claim 22 wherein the portion of the energy-activatable therapeutic agent is adapted to remain in the unactivated state in situ for at least one week.

24. The stent of claim 22 wherein the portion of the energy-activatable therapeutic agent is adapted to remain in the unactivated state in situ for at least one month.

25. A system for treating a subject, the system comprising:
a stent device comprising a selectively activatable treatment agent that, when activated, ablates target tissue proximate the stent device; and
an activation system configured to output a sufficient amount of energy to activate a therapeutically effective amount of the treatment agent when the stent device is positioned in situ.

26. The system of claim 25 wherein the activation system is an intraluminal catheter having an energy emission system that outputs sufficient energy to activate the treatment agent.

27. The system of claim 25 wherein the activation system comprises an energy emission system and a power supply in electrical communication with the energy emission system, the energy emission system is capable of delivering a selected amount of energy to the stent when the stent is implanted in a body lumen of the subject and the energy emission system is positioned outside the body of the subject.

28. The system of claim 27 wherein the energy emission system outputs light that activates the treatment agent carried by an elongate tubular covering of the stent device.

29. A method for treating a body lumen in a subject, the method comprising:
implanting a stent in the body lumen, the stent comprising:
a stent structure dimensioned to closely fit within the body lumen; and
a treatment agent coupled to the stent structure;
applying energy to the subject; and
activating a therapeutically effective amount of the treatment agent with the applied energy.

30. The method of claim 29 wherein the treatment agent is coupled to the stent structure by a linking moiety.

31. The method of claim 29 wherein activating the therapeutically effective amount of the treatment agent comprises:
supplying a predetermined amount of applied energy, the treatment agent activatable in part by at least one absorption wavelength or waveband, the applied energy comprising at least one wavelength or waveband capable of activating the treatment agent.

32. The method of claim 29, further comprising:
stopping the delivery of applied energy to the subject after activating the therapeutically effective amount of the treatment agent with the applied energy.

33. The method of claim 29 wherein, after implanting the stent, the therapeutically effective amount of the treatment agent is activated.

34. The method of claim 29, further comprising:
at a later time, reapplying energy to the subject to activate another therapeutically effective amount of the treatment agent.

35. The method of claim 34 wherein the later time is at least one day.

36. The method of claim 34 wherein the later time is at least one month.

37. The method of claim 29, further comprising:
viewing one or more markers of the implanted stent before activating the therapeutically effective amount of the treatment agent with the applied energy.

38. A stent device comprising:
an implantable body configured for placement in a body lumen; and
a treatment agent coupled to the implantable body, the treatment agent comprising a sufficient amount of an energy-activatable agent for effectively treating target tissue of the body lumen without being released in appreciable amounts when activated by applied energy.

39. The stent device of claim 38, further comprising:
one or more markers viewable under fluoroscopy for determining the position of the stent device relative to the body lumen.
